(19) United States
(12) Plant Patent Application Publication
(10) Pub. No.: US 2018/0160580 P1
(43) Pub. Date: Jun. 7, 2018

(54) Apple tree named 'Bay 4210'

(50) ;

(71) Applicant: Michael Neumüller, Hallbergmoos (DE)

(72) Inventor: Michael Neumüller, Hallbergmoos (DE)

(21) Appl. No.: 15/732,597

(22) Filed: Nov. 30, 2017

Related U.S. Application Data
(60) Provisional application No. 62/497,759, filed on Dec. 1, 2016.

(51) Int. Cl. A01H 5/08

(52) U.S. Cl. PLT/172

(57) ABSTRACT

A new and distinct Malus domestica (Borkh.) variety that bears fruits having a yellow skin color and good eating and keeping qualities.

### CROSS REFERENCE TO RELATED APPLICATION

This application claims the benefit of U.S. Provisional Patent Application Ser. No. 62/497,759, filed on Dec. 1, 2016, and entitled “Variety of Apple Tree Named ‘Bay 4210’”, the entire disclosure of which is incorporated herein by reference.

Botanical classification: Malus domestica (Borkh.).

Varietal denomination: ‘Bay 4210’.

### BACKGROUND OF THE INVENTION

The present invention comprises a new and distinct variety of apple tree, botanically known as Malus domestica (Borkh.), and referred to by the varietal name ‘Bay 4210’.

The new variety is the result of a planned breeding program conducted by the inventor in Hallbergmoos, Germany with the purpose of providing a new apple variety that exhibits tolerance to apple scab disease and good eating and keeping qualities. The new variety is the result of a cross between ‘Pinova’ (female parent, U.S. Plant Pat. No. 11,601) and ‘Topaz’ (male parent, unpatented). ‘Bay 4210’ was first asexually reproduced in Hallbergmoos, Germany by grafting in 2010.

The new variety is similar to ‘Pinova’ in its creamy yellow flesh color, but differs from ‘Pinova’ in skin color (yellow for ‘Bay 4210’ vs. red with yellow and pink for ‘Pinova’). The new variety is similar to ‘Topaz’ in fruit shape and growth habit, but differs from ‘Topaz’ in aroma and skin color (yellow for ‘Bay 4210’ vs. red with yellow and pink for ‘Topaz’). When compared to other apple varieties known to the breeder, the new variety generally differs in its bright yellow skin color at picking maturity.

The new variety has been trial and field tested and has been found to retain its distinctive characteristics and remain true to type through successive asexual propagations.

### DESCRIPTION OF THE DRAWINGS

The accompanying photographic drawings illustrate the new cultivar at approximately 5-6 years of age, with the color being as nearly true as possible with color illustrations of this type. It should be noted that colors may vary with growing conditions and time of year:

FIG. 1 illustrates multiple trees of the new variety;

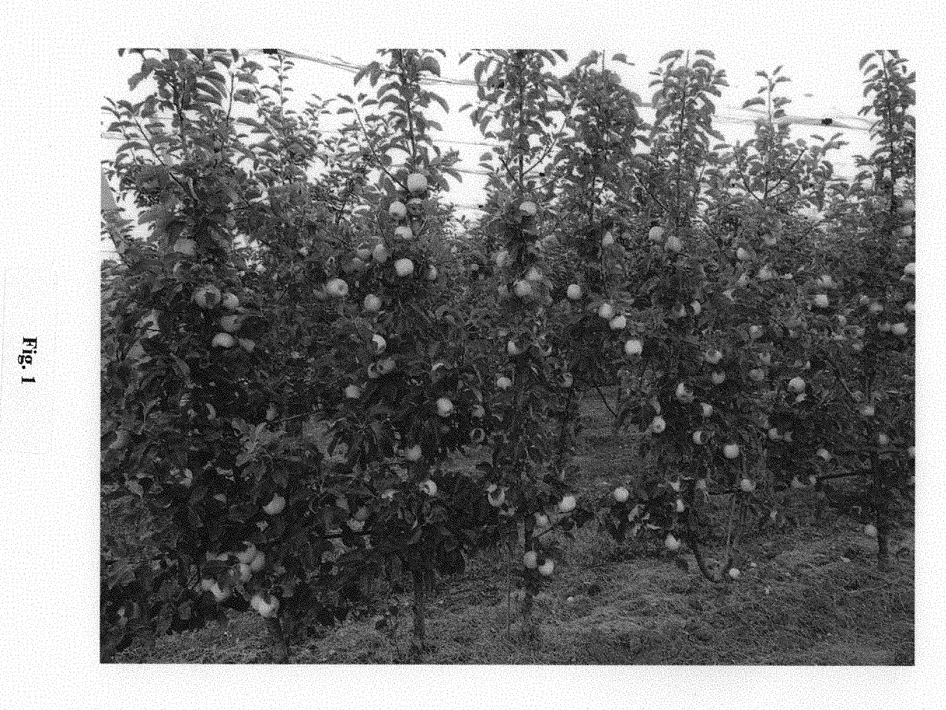

FIG. 2 illustrates a close-up view of the mature fruit of the new variety; and

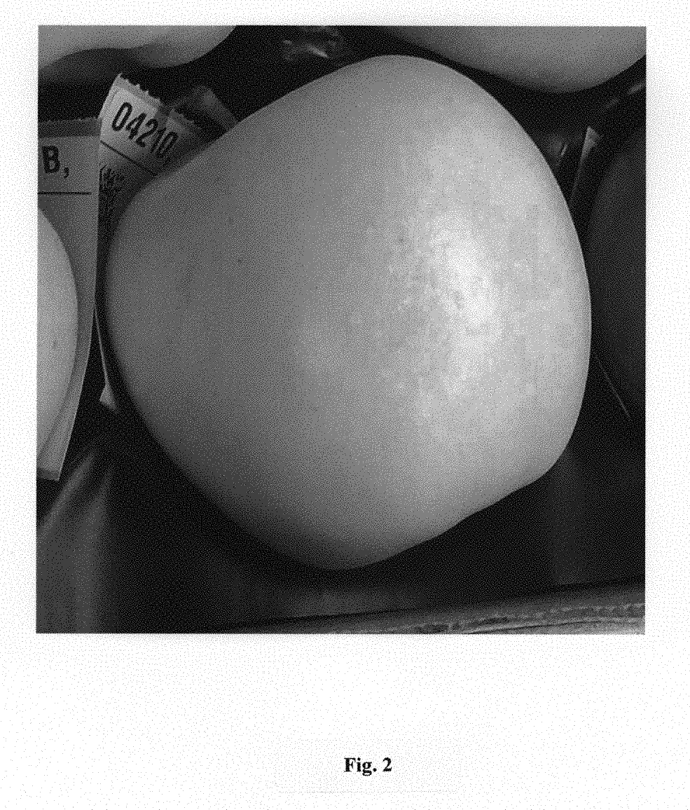

FIG. 3 illustrates a close-up view of the mature fruit of the new variety when cut in half

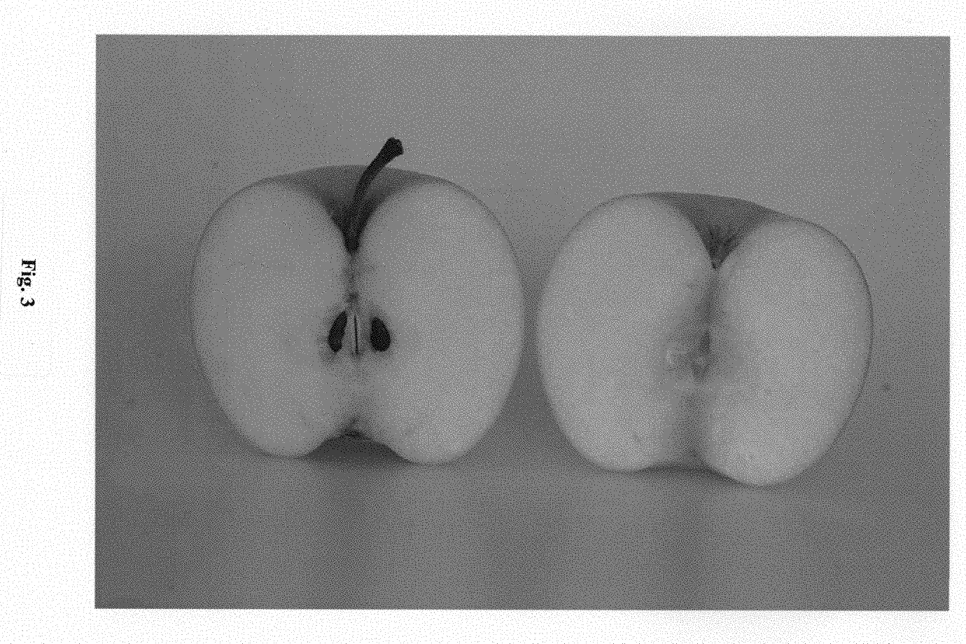

### DESCRIPTION OF THE PLANT

The following detailed description sets forth characteristics of the new cultivar. The new variety was grown under natural field conditions in Hallbergmoos, Germany. Color references are primarily to The R.H.S. Colour Chart of The Royal Horticultural Society of London, and were identified under natural light.
Tree:
   Age.—Approximately 5-6 years.
   Size.—Crown height of 2.5 m.
   Vigor.—Moderate.
   Production.—Highly productive.
   Growth type.—Upright and slightly spreading.
   Bearing.—Annual.
   Fruit.—Maturity when described: Full eating maturity.
   Skin.—Color: Yellow.
   Flesh.—Aroma: Citrus-like. Color: Creamy yellow.
   Keeping quality.—High.
   Tree winter hardiness.—High.
   Bud winter hardiness.—High.
   Disease resistance.—Tolerant to apple scab (caused by Venturia inaequalis).

I claim:

1. A new and distinct variety of Malus domestica (Borkh.) apple tree plant substantially as is herein described and illustrated.
